(19) United States
(12) Patent Application Publication
(10) Pub. No.: US 2005/0181488 A1
(43) Pub. Date: Aug. 18, 2005

(54) Method for producing L-threonine using bacteria belonging to the genus Escherichia

(76) Inventors: Valery Zavenovich Akhverdian, Moscow (RU); Ekaterina Alekseevna Savrasova, Moscow (RU); Natalia Nikolaevna Samsonova, Moscow (RU); Vladimir Yurievich Ermishev, Moscow (RU); Irina Borisovna Altman, Moscow (RU); Leonid Romanovich Ptitsyn, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/052,140

(22) Filed: Feb. 8, 2005

Related U.S. Application Data
(60) Provisional application No. 60/601,144, filed on Aug. 13, 2004.

(30) Foreign Application Priority Data
Feb. 12, 2004 (RU) 2004103986

(51) Int. Cl. C12P 13/04; C12P 13/08; C12N 15/74; C12N 1/21

(52) U.S. Cl. 435/106; 435/252.33; 435/488

(57) ABSTRACT

A method is disclosed for producing L-threonine using bacterium belonging to the genus Escherichia, wherein the bacterium has L-threonine productivity and has been modified to have enhanced expression of one or more of the following genes: glk, pgi, pfkA,, tpiA, gapA, pgk, eno, and pykA, which code for enzymes of glycolytic pathway.

This application claims priority under 35 U.S.C. §119(e) to provisional application 60/601,144, filed on Aug. 13, 2004.

### FIELD OF THE INVENTION

The present invention relates to biotechnology, specifically to a method for producing L-amino acids by fermentation, and more specifically to genes derived from the bacterium Escherichia coli. The genes are useful for improvement of L-amino acid productivity, for example, productivity of L-threonine.

### BRIEF DESCRIPTION OF THE RELATED ART

Conventionally, L-amino acids have been industrially produced by methods of fermentation utilizing strains of microorganisms obtained from natural sources or mutants of the same, especially modified to enhance L-amino acid productivity.

Enhancement of L-amino acid productivity has been accomplished, for example, by amplification of biosynthetic genes by transformation of a microorganism with recombinant DNA (see, for example, U.S. Pat. No. 4,278,765). These techniques are based on increasing the activities of the enzymes involved in amino acid biosynthesis, and/or desensitizing the target enzymes to feedback inhibition by the produced L-amino acid or its by-products (see, for example, U.S. Pat. Nos. 4,346,170, 5,661,012 and 6,040,160).

Various strains used for production of L-threonine by fermentation are known. There are strains with increased activities of the enzymes involved in L-threonine biosynthesis (U.S. Pat. Nos. 5,175,107; 5,661,012; 5,705,371; 5,939,307; EP0219027), strains resistant to some chemicals such as L-threonine and its analogs (WO0114525A1, EP301572A2, US 5,376,538), strains with the target enzymes desensitized to feedback inhibition by the produced L-amino acid of its by-products (U.S. Pat. Nos. 5,175,107; 5,661,012), strains with inactivated threonine degradation enzymes (U.S. Pat. Nos. 5,939,307; 6,297,031).

The known threonine-producing strain VKPM B-3996 (U.S. Pat. Nos. 5,175,107, and 5,705,371) is currently the best known threonine producer. For construction of the strain VKPM B-3996, several mutations and a plasmid, described below, were introduced in the parent strain E. coli K-12 (VKPM B-7). Mutant thrA gene (mutation thrA442) encodes aspartokinase homoserine dehydrogenase I, which imparts resistance to feedback inhibition by threonine. Mutant ilvA gene (mutation ilvA442) encodes threonine deaminase which has a low activity, leading to a low rate of isoleucine biosynthesis and a leaky phenotype of isoleucine starvation. In bacteria with ilvA442 mutation, transcription of thrABC operon is not repressed by isoleucine and therefore is very efficient for threonine production. Inactivation of tdh gene leads to prevention of the threonine degradation. The genetic determinant of saccharose assimilation (scrKYABR genes) was transferred to said strain. To increase expression of genes controlling threonine biosynthesis, plasmid pVIC40 containing mutant threonine operon thrA442BC was introduced in the intermediate strain TDH6. The amount of L-threonine accumulated during fermentation of the strain reaches up to 85 g/l.

The present inventors obtained, with respect to E. coli K-12, a mutant having a mutation thrR (herein referred to as rhtA23) that imparts resistance to high concentrations of threonine or homoserine in a minimal medium (Astaurova, O. B. et al., Appl. Bioch. And Microbiol., 21, 611-616 (1985)). This mutation also improved the production of L-threonine (SU Patent No. 974817), homoserine and glutamate (Astaurova, O. B. et al., Appl. Bioch. And Microbiol., 27, 556-561, 1991, EP 1013765 A) by the respective E. coli producing strain, such as the strain VKPM-3996. Furthermore, the present inventors have revealed that the rhtA gene exists at 18 min on E. coli chromosome close to the glnHPQ operon that encodes components of the glutamine transport system, and that the rhtA gene is identical to ORF 1 (ybiF gene, numbers 764 to 1651 in the GenBank accession number AAA218541, gi:440181), located betweenpexB and ompXgenes (US Patent Application Publication Nos. 2003/148473, 2003/157667). The unit expressing a protein encoded by the ORF 1 has been designated as rhtA (rht: resistance to homoserine and threonine) gene. Also, the present inventors have found that the rhtA23 mutation is an A-for-G substitution at position -1 with respect to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457, EP 1013765 A).

Under conditions whereby the mainstream threonine biosynthetic pathway is studied and optimized to a great extent, the further improvement of threonine-producing strains can be done by improving the efficiency of the central metabolism pathways, such as glycolysis (Embden-Meyerhof pathway), which generates energy and various precursors of metabolites.

The glycolytic pathway includes the following enzymes: glucokinase (EC 2.7.1.2) coded by glk gene, phosphoglucose isomerase (EC 5.3.1.9) coded by pgi gene, phosphofructokinase-1 (fructose-6-P 1-kinase) (EC 2.7.1.11) coded by pfkA gene, fructose-1,6-bisphosphate aldolase (EC 4.1.2.13) coded by fbaA gene, triose-phosphate isomerase (EC 5.3.1.1) coded by tpiA gene, glyceraldehyde-3-phosphate dehydrogenase (EC 1.2.1.12) coded by gapA gene, phosphoglycerate kinase (EC 2.7.2.3) coded by pgk gene, phosphoglycerate mutase (EC 2.7.5.3) coded by gpmA gene, enolase (EC 4.2.1.11) coded by eno gene and isoenzymes of pyruvate kinase (EC 2.7.1.40) coded by pykA and pykF genes (Escherichia coli and Salmonella, Second Edition, Editor in Chief: F. C. Neidhardt, ASM Press, Washington D.C., 1996).

The process for production of L-lysine or other feed additives containing L-lysine by fermentation of coryneform bacteria in which alleles of the endogenous glk gene are overexpressed under conditions suitable for the formation of the glk gene product glucokinase has been disclosed (PCT application WO03054198A1). A method for the production of shikimic acid and derivatives thereof by E. coli having the ability to convert the carbon source to shikimic acid and transformed with recombinant DNA comprising a gene encoding a glucose facilitator protein and a glucokinase from Zymomonas mobilis has also been disclosed (PCT application WO0229078A2). Also, processes for the microbial preparation of intracellular metabolic intermediates, in particular erythrose 4-phosphate, and alternative processes for the microbial preparation of substances, in particular aromatic amino acids such as L-phenylalanine, in which the activity of a transaldolase is increased in a microorganism producing these substances has been disclosed (U.S. Pat. No. 6,316,232). The '232 patent discloses as preferred embodiments that the activity of a transketolase or the activity of a transport protein for the PEP-independent uptake of a sugar and/or the activity of a glucokinase are/is additionally increased. Microorganisms employed include those belonging to the genus Escherichia, Serratia, Bacillus, Corynebacterium or Breibacterium.

A method for producing L-amino acids, particularly L-lysine, comprising culturing an altered bacterial cell, particularly Corynebacterium glutamicum, having an increased amount of NADPH as compared to an unaltered bacterial cell, wherein said altered bacterial cell has increased carbon flux through the oxidative branch of the pentose phosphate pathway have been disclosed (PCT application WO0107626A2). This publication discloses as the preferred embodiment of the method an altered bacterial cell which has decreased carbon flux via the glycolytic pathway due to a decreased amount of 6-phosphoglucose isomerase enzymatic activity, which results from a mutation in the pgi gene. A similar method for producing L-lysine using coryneform bacteria having intracellular activity of a phosphoglucose isomerase (pgi) enzyme eliminated has also been disclosed (U.S. Pat. No. 6,586,214).

A method for producing L-lysine comprising cultivating L-lysine-producing coryneform bacterium in which the intracellular activity of 6-phosphofructokinase coded by pfkA gene is increased has been disclosed (European patent application EP119543 IA1). At the same time, a process for the fermentative preparation of L-amino acids, in particular L-lysine, comprising culturing coryneform bacteria to produce the desired L-amino acid, in which at least the gene coding for 6-phosphofructokinase (pfkA gene) and/or the gene coding for 1-phosphofructokinase (fruK gene) are/is attenuated has been disclosed (PCT application WO02074944A1). This publication discloses as the preferred embodiment of the process the preparation of L-lysine using coryneform bacterium in which, in addition to attenuation of pfkA and/or fruK genes, one or more of the genes selected from the group comprising the lysC gene coding for a feedback-resistant aspartate kinase, the dapA gene coding for dihydrodipicolinate synthase, the gap gene coding for glyceraldehyde phosphate dehydrogenase, the pyc gene coding for pyruvate carboxylase, the mqo gene coding for malate:quinone oxidoreductase, the zwf gene coding for glucose phosphate dehydrogenase, the lysE gene coding for lysine exporter, the zwal gene coding for the zwal protein, the gene tpi coding for triose phosphate isomerase, and the pgk gene coding for 3-phosphoglycerate kinase is/are simultaneously enhanced, and, in particular, overexpressed.

A process for the preparation of L-amino acids, such as L-lysine and L-threonine, by fermentation of coryneform bacteria, in which at least the zwf gene is amplified, has been described (PCT application WO0170995A1). This publication discloses as the preferred embodiment of the process the preparation of the L-amino acids using coryneform bacterium in which, in addition to attenuation of zwf gene, one or more of the genes selected from the group comprising dapA gene which codes for dihydrodipicolinate synthase, the lysC gene which codes for a feed back resistant aspartate kinase, the gap gene which codes for glycerolaldehyde-3-phosphate dehydrogenase, the pyc gene which codes for pyruvate carboxylase, the tkt gene which codes for transketolase, the gnd gene which codes for gluconate 6-phosphate dehydrogenase, the lysE gene which codes for lysine exporter, the zwal gene, the eno gene which codes for enolase is/are amplified or over-expressed at the same time.

Coryneform bacteria which produce L-amino acids, including L-threonine, having at least one copy presented at the natural site (locus), and up to three additional copies of open reading frames (ORF) chosen from a group including, among others, eno, gap, pgk and tpi genes, have been disclosed (PCT applications WO03014330A2, WO03040373A2).

A process for preparing L-glutamic acid by fermentation of coryneform bacteria in which the nucleotide sequence coding for D-alanine racemase (alr) is attenuated, and in particular, eliminated, has been disclosed (PCT application WO0208437A2). This publication discloses as the preferred embodiment of the process the preparation of L-glutamic acid using coryneform bacterium in which, in addition to attenuation of the nucleotide sequence coding for D-alanine racemase (alr), one or more of the genes selected from the group including, among others, the gap and eno genes are enhanced, and in particular, over-expressed.

A method for producing fine chemicals or metabolites, such as L-threonine, using microorganisms, in particular, coryneform bacteria or Escherichia coli, in which the phosphorylatability of at least one protein has been permanently altered such that the biosynthesis of at least one fine chemical synthesized by the microorganism is increased compared to the wild-type due to a mutation in at least one amino acid of the protein, has been disclosed (PCT application WO03023016A2). One example of such protein is mutant enolase (enoS330E).

Among genes coding for enzymes of glycolytic pathway, four genes have been used for improvement of L-threonine production using bacterium of Enterobacteriaceae family, particularly Escherichia coli. There are fbaA, gpmA (pgm), pykF and pfkB genes.

Thus, process for the preparation of L-amino acids, in particular L-threonine, by fermentation of Enterobacteriaceae family microorganisms which produce the desired L-amino acid and in which the fba gene or the nucleotide sequence which codes for this gene is enhanced, in particular, over-expressed, has been disclosed (PCT application WO03004664A2). At the same time, a process for the fermentative preparation of L-amino acids, in particular L-threonine, by fermentation of microorganisms of the Enterobacteriaceae family which produce the desired L-amino acid and in which one or more of the genes chosen from the group comprising, among others, fba gene or nucleotide sequences which code for these, is/are attenuated, in particular eliminated, has been disclosed by the same applicant in the PCT application WO03004662A2, but it contains no examples.

Also, a process for the preparation of L-amino acids, in particular L-threonine, by fermentation of microorganisms of the Enterobacteriaceae family which produce the desired L-amino acid and in which the pgm gene or the nucleotide sequence which codes for this is enhanced, in particular, over-expressed, has been disclosed (PCT application WO03004598A2). At the same time, the process for the fermentative preparation of L-amino acids, in particular L-threonine, by fermentation of microorganisms of the Enterobacteriaceae family which produce the desired L-amino acid and in which one or more of the genes chosen from the group comprising among others pgm gene or nucleotide sequences which code for these is/are attenuated, in particular, eliminated, has been disclosed by the same applicant in the PCT application WO03004662A2, but it contains no examples.

And, the process for the preparation of L-amino acids, in particular L-threonine, by fermentation of microorganisms of the Enterobacteriaceae family which produce the desired L-amino acid and in which the pykF gene or the nucleotide sequence which codes for this is enhanced, in particular, over-expressed, has been disclosed (PCT application WO03008609A2). At the same time, a process for the fermentative preparation of L-amino acids, in particular L-threonine, by fermentation of microorganisms of the Enterobacteriaceae family which produce the desired L-amino acid and in which one or more of the genes chosen from the group comprising, among others, pykF gene or nucleotide sequences which code for these is/are attenuated, in particular, eliminated, has been disclosed by the same applicant in the PCT application WO03008600A2, but it contains no examples.

And finally, a process for the preparation of L-amino acids, in particular L-threonine, by fermentation of microorganisms of the Enterobacteriaceae family which produce the desired L-amino acid and in which the pfkB gene or the nucleotide sequence which codes for this is enhanced, in particular over-expressed, was disclosed (PCT application WO03008610A2). At the same time, process for the fermentative preparation of L-amino acids, in particular L-threonine, by fermentation of microorganisms of the Enterobacteriaceae family which produce the desired L-amino acid and in which one or more of the genes chosen from the group comprising among others pfkB gene or nucleotide sequences which code for these, is (are) attenuated, in particular eliminated, was claimed by the same applicant in the PCT application WO03008600A2 containing no examples.

There have been no disclosures to date of using bacterium belonging to the genus Escherichia with enhanced expression of genes coding for enzymes of glycolytic pathway, such as glk, pgi, pfkA, tpiA, gapA, pgk, eno and pykA, for production of L-threonine.

### SUMMARY OF THE INVENTION

An object of present invention is to enhance the productivity of L-threonine-producing strains and to provide a method for producing L-threonine using these strains.

This aim was achieved by the finding that genes, such as glk, pgi, pfkA, tpiA, gapA, pgk, eno and pykA, which code for enzymes of the glycolytic pathway, upon being cloned on a low copy vector, enhance L-threonine production of L-threonine-producing strains when the strain is transformed with the plasmid harboring the gene. Thus the present invention has been completed.

It is an object of the invention to provide an L-threonine-producing bacterium belonging to the genus Escherichia, wherein the bacterium has been modified to enhance an activity of one or more of glycolytic enzymes.

It is a further object of the invention to provide an L-threonine producing bacterium belonging to the genus Escherichia, wherein the bacterium has been modified to enhance expression of one or more of the genes chosen from the group consisting of glk, pgi, pfkA, tpiA, gapA, pgk, eno and pykA, which code for enzymes of glycolytic pathway, or the nucleotide sequences, which code for these.

It is a further object of the invention to provide the bacterium as described above, wherein the expression of one or more of the genes is enhanced by increasing copy number of the gene(s), or modifying an expression control sequence of the gene(s) so that the expression of the gene(s) is enhanced.

It is a further object of the invention to provide the bacterium as described above, wherein the copy number is increased by transformation of the bacterium with a low copy vector containing the gene or genes.

It is a further object of the invention to provide the bacterium as described above, wherein the genes are originated from a bacterium belonging to the genus Escherichia.

It is a further object of the invention to provide the bacterium as described above, wherein the bacterium has been further modified to enhance expression of one or more genes selected from the group consisting of:
the mutant thrA gene which codes for aspartokinase homoserine dehydrogenase I resistant to feed back inhibition by threonine;
the thrB gene which codes for homoserine kinase;
the thrC gene which codes for threonine synthase;
the rhtA gene, which codes for putative transmembrane protein.

It is a further object of the invention to provide the bacterium as described above, wherein the bacterium has been modified to increase expression amount of the mutant thrA gene, the thrB gene, the thrC gene and the rhtA gene.

And it is a further object of the invention to provide a method for producing L-threonine, which comprises cultivating the bacterium as described above in a culture medium to produce and accumulate L-threonine in the culture medium, and collecting the L-threonine from the culture medium.

### BRIEF DESCRIPTION OF THE DRAWINGS

FIG. 1 shows the structure of synthetic mutant promoter P_{A3m}.

### DESCRIPTION OF THE PREFERRED EMBODIMENTS

The bacterium of the present invention is an L-threonine-producing bacterium belonging to the genus Escherichia, wherein the bacterium has been modified to enhance an activity of one or more of the glycolytic enzymes. Particularly, the bacterium of the present invention is an L-threonine-producing bacterium belonging to the genus Escherichia, wherein the bacterium has been modified to enhance expression of one or more of the genes chosen from the group consisting of glk, pgi, pfkA, tpiA, gapA, pgk, eno and pykA, which code for enzymes of glycolytic pathway, or the nucleotide sequences, which code for these.

In the present invention, “L-threonine-producing bacterium” means a bacterium, which has an ability to cause accumulation of L-threonine in a medium, when the bacterium of the present invention is cultured in the medium. The L-threonine-producing ability may be imparted or enhanced by breeding. The phrase “L-threonine-producing bacterium” as used herein also means a bacterium, which is able to produce and cause accumulation of L-threonine in a culture medium in amount larger than a wild type or parental strain of E. coli, such as E. coli K-12 strain.

The phrase “a bacterium belonging to the genus Escherichia” means that the bacterium is classified as the genus Escherichia according to the classification known to a person skilled in the art of microbiology. Examples of a microorganism belonging to the genus Escherichia as used in the present invention include, but are not limited to, Escherichia coli (E. coli).

The bacterium belonging to the genus Escherichia that can be used in the present invention is not particularly limited, however, for example, bacteria described by Neidhardt, F. C. et al. (Escherichia coli and Salmonella typhimurium, American Society for Microbiology, Washington D.C., 1208, Table 1) are encompassed by the present invention.

The phrase “modified to enhance expression of gene(s)” means that the expression amount of the gene(s) is higher than that of a non-modified strain, for example, a wild-type strain. Examples of such modifications include increasing the number of gene(s) to be expressed per cell, increasing the expression level of the gene, and so forth. The quantity of the copy number of the expressed gene is measured, for example, by restriction of the chromosomal DNA, followed by Southern blotting using a probe constructed based on the gene sequence, fluorescence in situ hybridization (FISH), and the like. The level of gene expression is measured by different methods, including Northern blotting, quantitative RT-PCR and the like. Furthermore, the Escherichia coli K-1, for example, can be used as a wild-type strain for comparison. As a result of the enhancement of expression of the genes(s), the amount of L-threonine which accumulates in a medium increases.

Enzymes of glycolytic pathway according to the present invention are presented by glucokinase (EC 2.7.1.2) coded by glk gene, phosphoglucose isomerase (EC 5.3.1.9) coded by pgi gene, phosphofructokinase-1 (fructose-6-P 1-kinase) (EC 2.7.1.11) coded by pfkA gene, triose-phosphate isomerase (EC 5.3.1.1) coded by tpiA gene, glyceraldehyde-3-phosphate dehydrogenase (GAPDH) (EC 1.2.1.12) coded by gapA gene, phosphoglycerate kinase (EC 2.7.2.3) coded by pgk gene, enolase (EC 4.2.1.11) coded by eno gene and isoenzymes of pyruvate kinase (EC 2.7.1.40) coded by pykA gene.

In the present invention, the term “glucokinase” means an enzyme capable of catalyzing the reaction of ATP-dependent phosphorylation of glucose with formation of glucose-6-phosphate. The term “phosphoglucose isomerase” means an enzyme capable of catalyzing the reaction of conversion of glucose-6-phosphate into fructose-6phosphate. The term “phosphofructokinase- 1 (fructose-6-P 1-kinase)” means an enzyme capable of catalyzing the reaction of ATP-dependent phosphorylation of glucose-6-phosphate with formation of glucose-1,6-diphosphate. The term “triose-phosphate isomerase” means an enzyme which interconverts dihydroxyacetone phosphate and glyceraldehyde-3-phosphate. The term “glyceraldehyde-3-phosphate dehydrogenase” means an enzyme capable of catalyzing the reaction of NAD⁺-dependent conversion of glyceraldehyde-3-phosphate into 1,3-diphosphoglucerate. The term “phosphoglycerate kinase” means an enzyme capable of catalyzing the reaction of dephosphorylation of 1,3-diphosphoglucerate with release of 3-phosphoglucerate and ATP. The term “enolase” means an enzyme capable of catalyzing the reaction of dehydration of 2-phosphoglucerate with release of phosphoenolpyruvate. The term “pyruvate kinase” means an enzyme capable of catalyzing the reaction of formation of pyruvate from phosphoenolpyruvate with release of ATP.

Any genes derived from bacteria belonging to the genus Escherichia and genes derived from other bacteria such as coryneform bacteria, bacteria belonging to the genus Bacillus or the like can be used as the genes coding for the glycolytic enzymes. Among these, genes derived from bacteria belonging to the genus Escherichia are preferred.

As the gene coding for glucokinase of Escherichia coli, glk gene has been elucidated (nucleotide numbers 2506481 to 2507446 in the sequence of GenBank accession NC_{—}000913.1, gi:16130320; SEQ ID NO: 1). The glk gene is located between b2387 and b2389 ORFs on the chromosome of E. coli strain K12. As the gene coding for phosphoglucose isomerase of Escherichia coli, pgi gene has been elucidated (nucleotide numbers 4231337 to 4232986 in the sequence of GenBank accession NC_{—}000913.1, gi:16131851; SEQ ID NO: 3). The pgi gene is located between lysC gene and yjbE on the chromosome of E. coli strain K12. As the gene coding for phosphofructokinase-1 of Escherichia coli, pfkA gene has been elucidated (nucleotide numbers 4105132 to 4106094 in the sequence of GenBank accession NC_{—}000913.1, gi:16131754; SEQ ID NO: 5). The pfkA gene is located between yiiP ORF and sbp gene on the chromosome of E. coli strain K12. As the gene coding for triose-phosphate isomerase of Escherichia coli, tpiA gene has been elucidated (nucleotide numbers 4108320 to 4109087 in the sequence of GenBank accession NC_{—}000913.1, gi:16131757; SEQ ID NO: 9). The tpiA gene is located between cdh gene and yiiQ ORF on the chromosome of E. coli strain K12. As the gene coding for glyceraldehyde-3-phosphate dehydrogenase of Escherichia coli, gapA gene has been elucidated (nucleotide numbers 1860795 to 1861790 in the sequence of GenBank accession NC_{—}000913.1, gi:16129733; SEQ ID NO: 11). The gapA gene is located between yeaA and yeaA ORFs on the chromosome of E. coli strain K12. As the gene coding for phosphoglycerate kinase of Escherichia coli, pgk gene has been elucidated (nucleotide numbers 3069479 to 3070642 in the sequence of GenBank accession NC_{—}000913.1, gi:16130827; SEQ ID NO: 13). The pgk gene is located between fbaA and epd genes on the chromosome of E. coli strain K1 2. As the gene coding for enolase of Escherichia coli, eno gene has been elucidated (nucleotide numbers 2904665 to 2905963 in the sequence of GenBank accession NC_{—}000913.1, gi:16130686; SEQ ID NO: 17). The eno gene is located between b2778 ORF and pyrG gene on the chromosome of E. coli strain K12. As the gene coding for pyruvate kinase II of Escherichia coli, pykA gene has been elucidated (nucleotide numbers 1935673 to 1937115 and 1753722 to 1755134 in the sequence of GenBank accession NC_{—}000913.1, gi:16129807 and gi:16129632; SEQ ID NOs: 19 and 21, respectively). The pykA gene is located between yebK ORF and msbB gene on the chromosome of E. coli strain K12. Therefore, the aforementioned genes can be obtained by PCR (polymerase chain reaction; refer to White, T.J. et al., Trends Genet., 5,185 (1989)) utilizing primers prepared based on the reported nucleotide sequence of the genes.

The glk gene originated from Escherichia coli is exemplified by a DNA which encodes the following protein (A) or (B):

(A) a protein, which comprises the amino acid sequence shown in SEQ ID NO: 2; or

(B) a protein which comprises an amino acid sequence including deletion, substitution, insertion or addition of one or several amino acids in the amino acid sequence shown in SEQ ID NO: 2, and which has an activity of glucokinase.

The pgi gene originated from Escherichia coli is exemplified by a DNA which encodes the following protein (C) or (D):

(C) a protein, which comprises the amino acid sequence shown in SEQ ID NO: 4; or

(D) a protein which comprises an amino acid sequence including deletion, substitution, insertion or addition of one or several amino acids in the amino acid sequence shown in SEQ ID NO: 4, and which has an activity of phosphoglucose isomerase.

The pfkA gene originated from Escherichia coli is exemplified by a DNA which encodes the following protein (E) or (F):

(E) a protein, which comprises the amino acid sequence shown in SEQ ID NO: 6; or

(F) a protein which comprises an amino acid sequence including deletion, substitution, insertion or addition of one or several amino acids in the amino acid sequence shown in SEQ ID NO: 6, and which has an activity of phosphofructokinase-I (fructose-6-P 1-kinase).

The tpiA gene originated from Escherichia coli is exemplified by a DNA which encodes the following protein (G) or (H):

(G) a protein, which comprises the amino acid sequence shown in SEQ ID NO: 8; or

(H) a protein which comprises an amino acid sequence including deletion, substitution, insertion or addition of one or several amino acids in the amino acid sequence shown in SEQ ID NO: 8, and which has an activity of triose-phosphate isomerase.

The gapA gene originated from Escherichia coli is exemplified by a DNA which encodes the following protein (I) or (J):

(I) a protein, which comprises the amino acid sequence shown in SEQ ID NO: 10; or

(J) a protein which comprises an amino acid sequence including deletion, substitution, insertion or addition of one or several amino acids in the amino acid sequence shown in SEQ ID NO: 10, and which has an activity of glyceraldehyde-3-phosphate dehydrogenase.

The pgk gene originated from Escherichia coli is exemplified by a DNA which encodes the following protein (K) or (L):

(K) a protein, which comprises the amino acid sequence shown in SEQ ID NO: 12; or

(L) a protein which comprises an amino acid sequence including deletion, substitution, insertion or addition of one or several amino acids in the amino acid sequence shown in SEQ ID NO: 12, and which has an activity of phosphoglycerate kinase.

The eno gene originated from Escherichia coli is exemplified by a DNA which encodes the following protein (M) or (N):

(M) a protein, which comprises the amino acid sequence shown in SEQ ID NO: 14; or

(N) a protein which comprises an amino acid sequence including deletion, substitution, insertion or addition of one or several amino acids in the amino acid sequence shown in SEQ ID NO: 14, and which has an activity of enolase.

The pyk, gene originated from Escherichia coli is exemplified by a DNA which encodes the following protein (O) or (P):

(O) a protein, which comprises the amino acid sequence shown in SEQ ID NO: 16; or

(P) a protein which comprises an amino acid sequence including deletion, substitution, insertion or addition of one or several amino acids in the amino acid sequence shown in SEQ ID NO: 16, and which has an activity of pyruvate kinase.

The number of “several” amino acids differs depending on the position or the type of amino acid residues in the three dimensional structure of the protein. It may be, for example, 2 to 30, preferably 2 to 15, and more preferably 2 to 5 for the protein (A). This is because some amino acids have high homology to one another so the three dimensional structure of the protein or its activity is not affected by such change. Therefore, the protein (B) may be one which has homology of not less than 30 to 50 %, preferably 50 to 70 %, and more preferably between 70 to 90%, still more preferably greater than 90%, and most preferably greater than 95%, with respect to the entire amino acid sequence constituting glucokinase, and which has the activity of glucokinase. The same approach is applied to other proteins (C), (E), (G), (I), (K), (M) and (O).

The DNAs, which encodes for the substantially the same proteins as each of enzyme of glycolytic pathway described above, are obtained, for example, by modifying the nucleotide sequence of DNA encoding for the enzyme, for example, by means of the site-directed mutagenesis method so that one or more amino acid residues at a specified site involve deletion, substitution, insertion, or addition. DNA modified as described above is obtained by conventionally known mutation treatments. Such treatments include hydroxylamine treatment of the DNA encoding for proteins of present invention or treatment of the bacterium containing the DNA with UV irradiation or a reagent such as N-methyl-N′-nitro-N-nitrosoguanidine or nitrous acid.

A DNA encoding for substantially the same protein as glucokinase is obtained by expressing a DNA having the mutation as described above in an appropriate cell, and investigating the activity of any expressed product. A DNA coding for substantially the same protein as glucokinase can also be obtained by isolating a DNA that is hybridizable with a probe having a nucleotide sequence which contains, for example, the nucleotide sequence shown in SEQ ID NO: 1; under the stringent conditions, and codes for a protein having the activity of glucokinase, from DNA coding for glucokinase having a mutation or from a cell harboring it. The “stringent conditions” referred to herein are conditions under which so-called specific hybrids are formed, and non-specific hybrids are not formed. It is difficult to clearly express this condition by using any numerical value. However, for example, the stringent conditions can be exemplified by conditions under which DNAs having high homology, for example, DNAs having homology of not less than 50%, preferably 50 to 70%, and more preferably between 70 to 90%, still more preferably greater than 90%, and most preferably greater than 95%, are able to hybridize with each other, but DNAs having homology lower than the above are not able to hybridize with each other.

To evaluate degree of protein or DNA homology several calculation methods, such as BLAST search, FASTA search and CrustalW, can be used. BLAST (Basic Local Alignment Search Tool) is the heuristic search algorithm employed by the programs blastp, blastn, blastx, megablast, tblastn, and tblastx; these programs ascribe significance to their findings using the statistical methods of Karlin, Samuel and Stephen F. Altschul (“Methods for assessing the statistical significance of molecular sequence features by using general scoring schemes”. Proc. Natl. Acad. Sci. USA, 1990, 87:2264-68; “Applications and statistics for multiple high-scoring segments in molecular sequences”. Proc. Natl. Acad. Sci. USA, 1993, 90:5873-7). The FASTA search method is described by W. R. Pearson (“Rapid and Sensitive Sequence Comparison with FASTP and FASTA”, Methods in Enzymology, 1990 183:63-98). ClustalW method is described by Thompson J. D., Higgins D. G. and Gibson T. J. (“CLUSTAL W: improving the sensitivity of progressive multiple sequence alignment through sequence weighting, position-specific gap penalties and weight matrix choice”, Nucleic Acids Res. 1994, 22:4673-4680).

Alternatively, the stringent conditions may be exemplified by conditions under which DNAs are hybridized with each other at a salt concentration equivalent to ordinary washing conditions in Southern hybridization, i.e., 1×SSC, 0.1% SDS, preferably 0.1×SSC, 0.1% SDS, at 60° C. Duration of washing procedure depends on the type of membrane used for blotting and, as a rule, is recommended by manufacturer. For example, recommended duration of washing the Hybond™ N+ nylon membrane (Amersham) under stringent conditions is 15 minutes. Preferably, washing may be performed 2 to 3 times.

A partial sequence of the nucleotide sequence of SEQ ID NO: 1 can also be used as a probe. Probes may be prepared by PCR using primers produced based on the nucleotide sequence of SEQ ID NO: 1 as primers, and a DNA fragment containing the nucleotide sequence of SEQ ID NO: 1 as a template. When a DNA fragment having a length of about 300 bp is used as the probe, the washing conditions may include, for example, 50° C, 2×SSC and 0.1% SDS.

The substitution, deletion, insertion, or addition of nucleotides as described above also includes mutation, which naturally occurs (mutant or variant), for example, due to variety of species or genus of bacterium, which contains glucokinase.

A DNA coding for substantially the same proteins as the other enzymes of glycolytic pathway are obtained similarly to glucokinase as described above.

“Transformation of a bacterium with DNA encoding a protein” means introduction of the DNA into a bacterium cell, for example, by conventional methods. Transformation of a bacterial cell with this DNA will result in an increase in expression of the gene encoding the protein of present invention and will enhance the activity of the protein in the bacterial cell. Methods of transformation include any known methods that have hitherto been reported. For example, a method of treating recipient cells with calcium chloride so as to increase permeability of the cells to DNA has been reported for Escherichia coli K-12 (Mandel, M. and Higa, A., J. Mol. Biol., 53, 159 (1970)) and may be used.

Methods of gene expression enhancement include increasing the gene copy number. Introduction of a gene into a vector that is able to function in a bacterium belonging to the genus Escherichia increases the copy number of the gene. Preferably, low copy vectors are used. The low-copy vector is exemplified by pSC101, pMW118, pMW119 and the like. The term “low copy vector” is used for vectors, the copy number of which is up to 5 copies per cell.

Enhancement of gene expression may also be achieved by introduction of multiple copies of the gene into a bacterial chromosome by, for example, a method of homologous recombination, Mu integration or the like. For example, one round of Mu integration allows to introduce into bacterial chromosome up to 3 copies of the gene.

Enhancement of gene expression may also be achieved by modifying an expression control sequence of the gene so that the expression of the gene is enhanced, for example, by placing the DNA of the present invention under the control of a potent promoter. For example, the lac promoter, the trp promoter, the trc promoter, the P_{R} or the P_{L} promoters of lambda phage are known as potent promoters. Strength of promoter is defined by frequency of acts of the RNA synthesis initiation. Method for evaluation the strength of promoter described by, for example, Deuschle U., Kammerer W., Gentz R., Bujard H. (Promoters in Escherichia coli: a hierarchy of in vivo strength indicates alternate structures. EMBO J., 5, 2987-2994 (1986)).

Use of a potent promoter can be combined with multiplication of gene copies.

Alternatively, a promoter can be enhanced by, for example, introducing a mutation into the promoter to increase the transcription level of a gene located downstream of the promoter. Further, it is known that substitution of several nucleotides in a spacer between the ribosome binding site (RBS) and the start codon and especially the sequences immediately upstream of the start codon profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984). Earlier, the authors of present invention showed, the rhtA23 mutation is an A-for-G substitution at position -1 with respect to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that rhtA23 mutation enhances the rhtA gene expression and, as a consequence, increases the level of resistance to threonine, homoserine and some other substances transported out of cells.

Moreover, it is also possible to introduce nucleotide substitution into a promoter region of the one or more of genes of glycolysis on the bacterial chromosome so that it should be modified into a stronger one. The alteration of expression control sequence can be performed, for example, in the same manner as the gene substitution using a temperature sensitive plasmid, as disclosed in International Patent Publication WO00/18935 and Japanese Patent Publication No. 1-215280.

Increasing the copy number of one or more of genes coding for enzymes of glycolytic pathway can also be achieved by introducing multiple copies of the gene into chromosomal DNA of bacterium. To introduce multiple copies of a gene into a bacterial chromosome, homologous recombination is carried out by using a sequence whose multiple copies exist in the chromosomal DNA as targets. As sequences whose multiple copies exist in the chromosomal DNA, repetitive DNA, inverted repeats existing at the end of a transposable element can be used. Also, as disclosed in Japanese Patent Laid-open No. 2-109985, it is possible to incorporate the concrete gene into transposon, and allow it to be transferred to introduce multiple copies of the gene into the chromosomal DNA.

Methods for preparation of plasmid DNA, digestion and ligation of DNA, transformation, selection of an oligonucleotide as a primer and the like may be ordinary methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

The bacterium of the present invention can be obtained by introduction of the aforementioned DNAs into bacterium which inherently has the ability to produce L-threonine. Alternatively, the bacterium of the present invention can be obtained by imparting an ability to produce L-threonine to the bacterium already containing the DNAs.

Examples of parent strains encompassed by the present invention include, but are not limited to, the threonine-producing bacteria belonging to the genus Escherichia such as E. coli strain TDH-6/pVIC40 (VKPM B-3996) (U.S. Pat. No. 5,175,107, U.S. Pat. No. 5,705,371), E. coli strain NRRL-21593 (U.S. Pat. No. 5,939,307), E. coli strain FERM BP-3756 (U.S. Pat. No. 5,474,918), E. coli strains FERM BP-3519 and FERM BP-3520 (U.S. Pat. No. 5,376,538), E. coli strain MG442 (Gusyatiner et al., Genetika (in Russian), 14, 947-956 (1978)), E. coli strains VL643 and VL2055 (EP 1149911 A) and the like may be used.

The strain TDH-6 is deficient in the thrC gene as well as being sucrose-assimilative, and the ilvA gene has a leaky mutation. This strain has a mutation in the rhtA gene, which imparts resistance to high concentrations of threonine or homoserine. The strain B-3996 (TDH-6/pVIC40) contains the plasmid pVIC40 which had been obtained by inserting thrA*BC operon including mutant thrA gene encoding aspartokinase homoserine dehydrogenase I which has substantially desensitized feedback inhibition by threonine into RSF1010-derived vector. The strain B-3996 was deposited on Nov. 19, 1987 in All-Union Scientific Center of Antibiotics (Nagatinskaya Street 3-A, 113105 Moscow, Russian Federation) on Apr. 7, 1987 under the accession number RIA 1867. The strain was also deposited in Russian National Collection of Industrial Microorganisms (VKPM) (Dorozhny proezd. 1, Moscow 113545, Russian Federation) under the accession number B-3996.

Preferably, the bacterium of the present invention is preferably further modified to enhance expression of one or more of the following genes along with one or more of genes coding for enzymes of glycolytic pathway:
the mutant thrA gene which codes for aspartokinase homoserine dehydrogenase I resistant to feed back inhibition by threonine;
the thrB gene which codes for homoserine kinase;
the thrC gene which codes for threonine synthase;

Another preferred embodiment of the present invention is the bacterium modified to enhance the rhtA gene which codes for a putative transmembrane protein in addition to enhancement of gene(s) coding for glycolytic enzyme(s). The most preferred embodiment of the present invention is a bacterium modified to increase expression of the gene(s) coding for glycolytic enzyme(s), the mutant thrA gene, the thrB gene, the thrC gene and the rhtA gene.

The method for producing L-threonine of the present invention includes the steps of cultivating the bacterium of the present invention in a culture medium, allowing L-threonine to accumulate in the culture medium, and collecting L-threonine from the culture medium.

In the present invention, the cultivation, the collection and purification of L-threonine from the medium and the like may be performed in a manner similar to the conventional fermentation method wherein L-threonine is produced using a microorganism.

A medium used for culture may be either a synthetic or natural medium, so long as the medium includes a carbon source and a nitrogen source and minerals and, if necessary, appropriate amounts of nutrients which the microorganism requires for growth. The carbon source may include various carbohydrates such as glucose and sucrose, and various organic acids. Depending on the mode of assimilation of the chosen microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate, and digested fermentative microorganism are used. As minerals, potassium monophosphate, magnesium sulfate, sodium chloride, ferrous sulfate, manganese sulfate, calcium chloride, and the like are used. As vitamins, thiamine, yeast extract and the like are used. Additional nutrients can be added to the medium if necessary. For example, if the microorganism requires isoleucine for growth (isoleucine auxotrophy), a sufficient amount of isoleucine can be added to the cultivation medium.

The cultivation is performed preferably under aerobic conditions such as a shaking culture, and stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the L-threonine in the liquid medium.

After cultivation, solids such as cells can be removed from the liquid medium by centrifugation or membrane filtration, and then L- threonine can be collected and purified by ion-exchange, concentration and crystallization methods.

Examples

The present invention will be more concretely explained below with reference to the following non-limited examples. The E. coli strain VKPM B-3996 (U.S. Pat. No. 5,175,107) was used as a parental strain to evaluate the effect of the amplification of genes coding for the enzymes of glycolytic pathway on L-threonine production.

The plasmid pMW119 and it derivatives are compatible with plasmid pVIC40 (replicon pRSF 1010), therefore the two plasmids pVIC40 and derivative of pMW 119 containing the gene coding for enzyme of glycolytic pathway could be maintained in the bacterium simultaneously. In the tables with results of fermentations, data from at least three independent experiments are presented.

Example 1: Cloning of glk gene from E. coli and effect of enhanced expression of glk gene on L-threonine production.

The glk gene was obtained by PCR using chromosomal DNA of the E. coli strain MG 1655 (VKPM B-6195) as the template and primers P1 (SEQ ID NO: 17) and P2 (SEQ ID NO: 18). The strain MG1655 is available from American Type Culture Collection (ATCC700926). Primer P1 contains recognition site of BamHI restrictases introduced in the 5′-end thereof. Primer P2 contains recognition site of SacI restrictases introduced in the 5′-end thereof. The obtained DNA fragment (968 bp) containing the glk gene was treated with BamHI and Sacd restrictases and cloned into the plasmid pMW 119 previously modified to substitute promoter P_{lac} by promoter P_{R} of the phage lambda and then treated with the same restrictases. Thus the plasmid pMW-P_{R}-glk containing the glk gene under the control of promoter P_{R} was constructed. Non-regulated high level of glk gene expression could be achieved using this plasmid.

The pMW-P_{R}-glk plasmid was introduced into the streptomycin-resistant threonine producer E. coli strain B-3996 (U.S. Pat. No. 5,175,107). Thus, the strain B-3996(PMW-P_{R}-glk) was obtained.

Both E. coli strains B-3996 and B-3996(pMW-P_{R}-glk) were grown for 18-24 hours at 37° C. on L-agar plates containing streptomycin (100 μg/ml) and ampicillin (100 μg/ml). To obtain seed culture, the strain was grown on a rotary shaker (250 rpm) at 32° C. for 18 hours in 20×200 mm test tubes containing 2 ml of L-broth with 4% glucose. Then, the fermentation medium was inoculated with 0.1 ml (5%) of seed material. The fermentation was performed in 2 ml of minimal medium for fermentation in 20×200 mm test tubes. Cells were grown for 24 hours at 32° C. with shaking at 250 rpm.

After cultivation, an accumulated amount of L-threonine in the medium was determined by TLC. Sorbfil plates (Stock Company Sorbopolymer, Krasnodar, Russia) were developed with a mobile phase: propan-2-ol:acetone:water:25% aqueous ammonia=25:25:7:6 (v/v). A solution (2%) of ninhydrin in acetone was used as a visualizing reagent. The results are presented in Table 1.

The composition of the fermentation medium (g/l) is as follows:

| | |
|---|---|
| Glucose | 40.0 |
| (NH₄)₂SO₄ | 10.0 |
| KH₂PO₄ | 1.0 |
| MgSO₄.7H₂O | 0.4 |
| FeSO₄.7H₂O | 0.02 |
| MnSO₄.5H₂O | 0.02 |
| Thiamine HCl | 0.0002 |
| Yeast extract | 1.0 |
| CaCO₃ | 20.0 |
| L-isoleucine | 0.05 |

Glucose and magnesium sulfate are sterilized separately. CaCO₃ dry-heat is sterilized at 180° C. for 2 h. The pH is adjusted to 7.0. Antibiotics are introduced into the medium after sterilization.

TABLE 1

| Strain | OD₅₆₀ | Threonine, g/l |
|---|---|---|
| B-3996 | 9.7 ± 0.1 | 14.3 ± 0.1 |
| B-3996(pMW-P_{R}-glk) | 9.6 ± 0.1 | 14.8 ± 0.1 |

As seen from the Table 1, the enhancement of glk gene expression improved L-threonine productivity of the strain B-3996.

Example 2: Cloning of pfkA gene from E. coli and effect of enhanced expression of pfkA gene on L-threonine production.

The pfkA gene was obtained by PCR using chromosomal DNA of the E. coli strain MG 1655 (VKPM B-6195) as the template and primers P3 (SEQ ID NO: 19) and P4 (SEQ ID NO: 20). Primer P3 contains recognition site of BamHI restrictases introduced in the 5′-end thereof. Primer P4 contains recognition site of SacI restrictases introduced in the 5′-end thereof. Obtained DNA fragment (987 bp) containing pfkA gene was directly cloning into vector pCR 2.1 (Invitrogen) by overnight ligation at +4° C. Then BamHI -SacI DNA fragment containing pfk gene was recloned into the plasmid pMW119 previously modified to substitute promoter P_{lac} by promoter P_{R} of the phage lambda and then treated with the BamHI and SacI restrictases. Thus, the plasmid PMW-P_{R}-pfkA containing the pfkA gene under the control of promoter Pwas constructed. Non-regulated high level of pfkA gene expression could be achieved using this plasmid.

The PMW-P_{R}-pfkA plasmid was introduced into the streptomycin-resistant threonine producer E. coli strain B-3996 (U.S. Pat. No. 5,175,107). Thus, the strain B-3996(pMW-P_{R}-pfkA) was obtained.

Accumulation of L-threonine by E. coli strains B-3996 and B-3996(PMW-P_{R}-pfkA) was evaluated as described above (see Example 1). The results are presented in Table 2.

TABLE 2

| Strain | OD₅₆₀ | Threonine, g/l |
|---|---|---|
| B-3996 | 9.7 ± 0.1 | 14.3 ± 0.1 |
| B-3996(pMW-P_{R}-pfkA) | 9.3 ± 0.1 | 14.4 ± 0.1 |

Since the effect of enhanced expression of pfkA gene on L-threonine production in test tube fermentation was not impressive, the batch fermentation was performed in laboratory fermenters having a capacity of 1.0 liter.

For that purpose, the E. coli strains VKPM-3996 and VKPM-3996(PM-P_{R}-pfkA) were grown during 18-24 hours at 37° C. on L-agar plates containing streptomycin (100 μg/ml). Then one loop of the cells was transferred to 50 ml of L-broth of the following composition: tryptone—10 g/l, yeast extract—5 g/l, NaCl—5 g/l. The cells (50 ml, OD₅₄₀ -2 o.u.) grown at 37° C. within 5 hours on shaker (240 rpm) was used for seeding 450 ml of the medium for fermentation. The batch fermentation was performed in laboratory fermenters having a capacity of 1.0 1 under aeration (1/1 vvm) with stirring at a speed of 1200 rpm at 37 ° C. The pH value was maintained automatically at 6.6 using 8% ammonia liquor. The results are presented in Table 3.

The composition of the fermentation medium (g/l):

| | |
|---|---|
| Glucose | 100.0 |
| NH₄Cl | 1.75 |
| KH₂PO₄ | 1.0 |
| MgSO₄.7H₂O | 0.8 |
| FeSO₄.7H₂O | 0.01 |
| MnSO₄.5H₂O | 0.01 |
| Mameno(TN) | 0.15 |
| Betaine | 1.0 |
| L-isoleucine | 0.2 |

Glucose and magnesium sulfate are sterilized separately. pH is adjusted to 6.6.

TABLE 3

| Strain | OD₅₆₀ (final) | Threonine, g/l |
|---|---|---|
| B-3996 | 39.9 ± 2.1 | 33.80 ± 3.1 |
| B-3996(pMW-P_{R}-pfkA) | 34.4 ± 1.5 | 39.57 ± 1.0 |

As seen from the Table 3, the enhancement of pfkA gene expression improved L-threonine productivity of the strain B-3996.

Example 3: Cloning of fbaA gene from E. coli and effect of enhanced expression of fbaA gene on L-threonine production.

The fbaA gene was obtained by PCR using chromosomal DNA of the E. coli strain MG 1655 (VKPM B-6195) as the template and primers P5 (SEQ ID NO: 21) and P6 (SEQ ID NO: 22). Primer P5 contains the recognition site of BamHI restrictases introduced in the 5′-end thereof. Primer P6 contains recognition site of SacI restrictases introduced in the 5′-end thereof. The obtained DNA fragment (1155 bp) containing fbaA gene was treated with BamHI and SacI restrictases and cloned into the plasmid pMW119 previously modified to substitute promoter P_{lac} by promoter P_{R} of the phage lambda and then treated with the same restrictases. Thus, the plasmid PMW-P_{R}-fbaA containing the fbaA gene under the control of promoter P_{R} was constructed. Non-regulated high level of fbaA gene expression could be achieved using this plasmid.

The pMW-P_{R}-fbaA plasmid was introduced into the streptomycin-resistant threonine producer E. coli strain B-3996 (U.S. Pat. No. 5,175,107). Thus, the strain B-3996(pMW-P_{R}-fbaA) was obtained.

Accumulation of L-threonine by E. coli strains B-3996 and B-3996(PMW-P_{R}-fbaA) was evaluated as described above (see Example 1). The results are presented in Table 4.

TABLE 4

| Strain | OD₅₆₀ | Threonine, g/l |
|---|---|---|
| B-3996 | 9.7 ± 0.1 | 14.3 ± 0.1 |
| B-3996(pMW-P_{R}-fbaA) | 9.3 ± 0.2 | 15.1 ± 0.5 |

As seen from the Table 4, the enhancement of fbaA gene expression improved L-threonine productivity of the strain B-3996.

Example 4: Cloning of tpiA gene from E. coli and effect of enhanced expression of tpiA gene on L-threonine production.

The tpiA gene was obtained by PCR using chromosomal DNA of the E. coli strain MG 1655 (VKPM B-6195) as the template and primers P7 (SEQ ID NO: 23) and P8 (SEQ ID NO: 24). Primer P7 contains recognition site of BamHI restrictases introduced in the 5′-end thereof. Primer P8 contains recognition site of SacI restrictases introduced in the 5′-end thereof. The obtained DNA fragment (774 bp) containing tpiA gene was treated with BamHI and SacI restrictases and cloned into the plasmid pMW119 previously modified to substitute promoter P_{lac} by promoter P_{R} of the phage lambda and then treated with the same restrictases. Thus, the plasmid PMW-P_{R}-tpiA containing the tpiA gene under the control of promoter P_{R} was constructed. Non-regulated high levels of tpiA gene expression could be achieved using this plasmid.

The PMW-P_{R}-tpiA plasmid was introduced into the streptomycin-resistant threonine producer E. coli strain B-3996 (U.S. Pat. No. 5,175,107). Thus, the strain B-3996(pMW-P_{R}-tpiA) was obtained.

Accumulation of L-threonine by E. coli strains B-3996 and B-3996(pMW-P_{R}-tpiA) was evaluated as described above (see Example 1). The results are presented in Table 5.

TABLE 5

| Strain | OD₅₆₀ | Threonine, g/l |
|---|---|---|
| B-3996 | 9.7 ± 0.1 | 14.3 ± 0.1 |
| B-3996(pMW-P_{R}-tpiA) | 9.6 ± 0.5 | 14.6 ± 0.1 |

Since the effect of enhanced expression of tpiA gene on L-threonine production in test tube fermentation was not impressive, the batch fermentation was performed in laboratory fermenters having a capacity of 1.0 liter as described above (see Example 2). The results are presented in Table 6.

TABLE 6

| Strain | OD₅₆₀ (final) | Threonine, g/l |
|---|---|---|
| B-3996 | 39.9 ± 2.1 | 33.80 ± 3.1 |
| B-3996(pMW-P_{R}-tpiA) | 34.9 ± 2.6 | 37.16 ± 1.4 |

As seen from the Table 6, the enhancement of tpiA gene expression improved L-threonine productivity of the strain B-3996.

Example 5: Cloning of gapA gene from E. coli and effect of enhanced expression of gapA gene on L-threonine production.

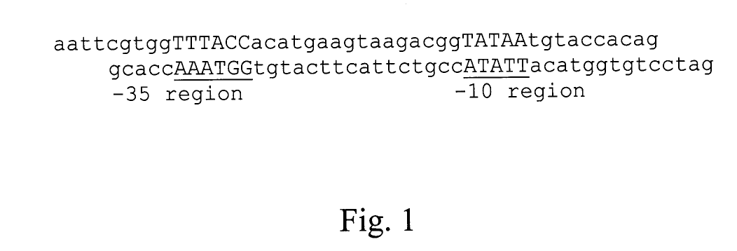

To study the effect of enhanced expression of gapA gene on L-threonine production, gapA gene was cloned into plasmid pMW119 under control of mutant synthetic promoter (A3m) derived from A3 promoter of coliphage T3 (Yamada, M. et al. Promoter sequence analysis in Bacillus and Escherichia coli: construction of strong promoter in E. coli. Gene, 99(1), 109-114 (1991)). The mutations introduced into the A3 promoter sequence led to decreased promoter strength of this constitutive polymerase holoenzyme Eσ⁷⁰—dependent promoter. Sequences of the oligonucleotides formed the mutant synthetic promoter A3m are shown in SEQ ID NO: 25 and 26. Structure of the promoter A3m is depicted on FIG. 1.

The gapA gene was obtained by PCR using chromosomal DNA of the E. coli strain MG 1655 (VKPM B-6195) as the template and primers gapA-5′(SEQ ID NO: 27) and gapA-ter (SEQ ID NO: 28). Primer gapA-5′ contains recognition site of BamHI restrictases introduced in the 5′-end thereof. Primer gapA-ter contains recognition site of XbaI restrictases introduced in the 5′-end thereof. Obtained DNA fragment (1.2 kbp) containing gapA gene with 5′-untranslated region up to P1 start transcription site and without own promoter's region was treated with BamHI and XbaI restrictases, ligated with synthetic A3m promoter having sticky ends of EcoRI and BamHI restriction sites and cloned into vector pMW119 previously treated with EcoRI and XbaI restrictases. Thus, the plasmid pMW119-PA3m-gapA was obtained. The structure of gapA gene was confirmed by sequencing.

E. coli cells HB101 were transformed with the plasmid pMW119-PA3m-gapA (Sambrook J. and Russell D. W. 2001. Molecular cloning: a laboratory manual, 3rd ed. Cold Spring Harbor Laboratory Press, Cold Spring Harbor, N.Y.). The strain MG1655 is available from American Type Culture Collection (ATCC33694). And activities of GAPDH at mid-log growth phase in strains HB101 and HB101 (pMW-PA3m-gapA) were determined according the procedure described in by Peng, L., and Shimizu, K. (Appl. Microbiol. Biotechnol. 61, 163-178 (2003)). The results are presented in Table 7.

TABLE 7

| Strain | Activity (nmol/mg * min) |
|---|---|
| HB101 | 39 |
| HB101(pMW-PA3m-gapA) | 86 |

As seen from Table 7, the HB101 cells harboring the plasmid possessed more than 2-time higher GAPDH activity.

Then the pMW-PA3m-gapA plasmid was introduced into the streptomycin-resistant threonine producer E. coli strain B-3996 (U.S. Pat. No. 5,175,107). Thus, the strain B-3996(pMW-PA3m-gapA) was obtained.

Both E. coli strains B-3996 and B-3996(pMW119-pA3-gapA) were grown overnight at 37° C. on LB-agar plates, or LB-agar plates containing ampicillin (100 μg/ml) in the case of the strain B-3996 (pMW119-pA3-gapA). One loop (OD595˜2-3 o.u.) of the night cell culture of each strains was transferred to 2 ml of minimal test tube fermentation medium of the following composition: yeast extract—2,0 g/l, (NH₄)₂SO₄—16.0 g/l, K₂HPO₄—0.7 g/l, MgSO₄.7H₂O—1.0 g/l, MnSO₄.5H₂O—0.01 g/l, FeSO₄.7H₂O—0.01 g/l, thiamine HCl (B₁)—0.2 mg/l, glucose—4 %, CaCO₃ (chalk)—30.0 g/l, L-isoleucine—50 mg/l, ampicillin—100 μg/ml (only in case of the strain 3996 (pMW119-pA3-gapA)). The cells were grown 48 h at 32° C. under permanent rotating (250 rpm).

After cultivation, the accumulated amount of L-threonine in the medium was determined by paper chromatography. The mobile phase has the following composition: n-butanol:acetic acid:water=4:1:1. The amino acids were colored by ethanol solution of ninhydrine (1%) containing CdCl₂ (0,5%). After 1 hour incubation at 37° C., the samples were measured at OD₅₀₈. The results are presented in Table 8.

TABLE 8

| Strain | OD₅₅₅ | Threonine, g/l |
|---|---|---|
| B-3996 | 13.2 ± 0.1 | 13.6 ± 0.2 |
| B-3996(pMW-PA3m-gapA) | 13.3 ± 0.1 | 15.6 ± 0.3 |

As seen from the Table 8, the enhancement of gapA gene expression improved L-threonine productivity of the strain B-3996.

Example 6: Cloning of eno gene from E. coli and effect of enhanced expression of eno gene on L-threonine production.

The eno gene was obtained by PCR using chromosomal DNA of the E. coli strain MG 1655 (VKPM B-6195) as the template and primers P9 (SEQ ID NO: 29) and P10 (SEQ ID NO: 30). Primer P9 contains recognition site of BamHI restrictases introduced in the 5′-end thereof. Primer P10 contains recognition site of SacI restrictases introduced in the 5′-end thereof. The obtained DNA fragment (1298 bp) containing the eno gene was treated with BamHI and SacI restrictases and cloned into the plasmid pMW119 previously modified to substitute promoter P_{lac} by promoter P_{R} of the phage lambda and then treated with the same restrictases. Thus, the plasmid PMW-P_{R}-eno containing the eno gene under the control of promoter P_{R} was constructed. Non-regulated high level of eno gene expression could be achieved using this plasmid.

The PMW-P_{R}-eno plasmid was introduced into the streptomycin-resistant threonine producer E. coli strain B-3996 (U.S. Pat. No. 5,175,107). Thus, the strain B-3996(pMW-P_{R}-eno) was obtained.

Accumulation of L-threonine by E. coli strains B-3996 and B-3996(pMW-P_{R}-eno) was evaluated as described above (see Example 1). The results are presented in Table 9.

TABLE 9

| Strain | OD₅₆₀ | Threonine, g/l |
|---|---|---|
| B-3996 | 9.7 ± 0.1 | 14.3 ± 0.1 |
| B-3996(pMW-P_{R}-eno) | 9.3 ± 0.2 | 14.9 ± 0.4 |

As seen from the Table 9, the enhancement of fbaA gene expression improved L-threonine productivity of the strain B-3996.

Example 7: Cloning of pgi gene from E. coli and effect of enhanced expression of pgi gene on L-threonine production.

The pgi gene was obtained by PCR using chromosomal DNA of the E. coli strain MG 1655 (VKPM B-6195) as the template and primers P11 (SEQ ID NO: 31) and P12 (SEQ ID NO: 32). Primer P11 contains recognition site of BamHI restrictases introduced in the 5′-end thereof. Primer P12 contains recognition site of SacI restrictases introduced in the 5′-end thereof. The obtained DNA fragment (1657 bp) containing pgi gene was treated with BamHI and SacI restrictases and cloned into the plasmid pMW119 previously modified to substitute promoter P_{lac} by promoter P_{R} of the phage lambda and then treated with the same restrictases. Thus, the plasmid pMW-P_{R}-pgi containing the pgi gene under the control of promoter P_{R} was constructed. Non-regulated high level of pgi gene expression could be achieved using this plasmid.

The pMW-P_{R}-pgi plasmid was introduced into the streptomycin-resistant threonine producer E. coli strain B-3996 (U.S. Pat. No. 5,175,107). Thus, the strain B-3996(pMW-P_{R}-pgi) was obtained.

Accumulation of L-threonine by E. coli strains B-3996 and B-3996(pMW-P_{R}-pgi) was evaluated as described above (see Example 1). The results are presented in Table 10.

TABLE 10

| Strain | OD₅₆₀ | Threonine, g/l |
|---|---|---|
| B-3996 | 8.7 ± 0.4 | 18.4 ± 0.7 |
| B-3996(pMW-P_{R}-pgi) | 8.4 ± 0.2 | 19.7 ± 0.4 |

As seen from Table 10, the enhancement of pgi gene expression improved L-threonine productivity of the strain B-3996.

Example 8: Cloning of pgk gene from E. coli and effect of enhanced expression of pgk gene on L-threonine production.

The pgk gene was obtained by PCR using chromosomal DNA of the E. coli strain MG 1655 (VKPM B-6195) as the template and primers P13 (SEQ ID NO: 33) and P14 (SEQ ID NO: 34). Primer P13 contains recognition site of BamHI restrictases introduced in the 5′-end thereof. Primer P14 contains recognition site of SacI restrictases introduced in the 5′-end thereof. The obtained DNA fragment (1163 bp) containing the pgk gene was treated with BamHI and SacI restrictases and cloned into the plasmid pMW119 previously modified to substitute promoter P_{lac} by promoter P_{R} of the phage lambda and then treated with the same restrictases. Thus, the plasmid pMW-P_{R}-pgk containing the pgk gene under the control of promoter P_{R} was constructed. Non-regulated high level of pgk gene expression could be achieved using this plasmid.

The pMW-P_{R}-pgi plasmid was introduced into the streptomycin-resistant threonine producer E. coli strain B-3996 (U.S. Pat. No. 5,175,107). Thus, the strain B-3996(pMW-P_{R}-pgk) was obtained.

Accumulation of L-threonine by E. coli strains B-3996 and B-3996(pMW-P_{R}-pgk) was evaluated as described above (see Example 1). The results are presented in Table 11.

TABLE 11

| Strain | OD₅₆₀ | Threonine, g/l |
|---|---|---|
| B-3996 | 10.3 ± 0.5 | 19.2 ± 0.2 |
| B-3996(pMW-P_{R}-pgk) | 10.2 ± 0.5 | 20.3 ± 0.4 |

As seen from the Table 11, the enhancement of pgk gene expression improved L-threonine productivity of the strain B-3996.

While the invention has been described in detail with reference to preferred embodiments thereof, it will be apparent to one skilled in the art that various changes can be made, and equivalents employed, without departing from the scope of the invention. Each of the aforementioned documents, including a priority application of Russian patent application 2004103986 filed on Feb. 12, 2004, is incorporated by reference herein in its entirety.

1. An L-threonine-producing bacterium belonging to the genus Escherichia, wherein the bacterium has been modified to enhance an activity of one or more of the glycolytic enzymes.

2. An L-threonine-producing bacterium belonging to the genus Escherichia, wherein the bacterium has been modified to enhance expression of one or more of the genes chosen from the group consisting of glk, pgi, pfk A, tpiA, gapA, pgk, eno and pykA, which code for enzymes of the glycolytic pathway, or the nucleotide sequences, which code for these.

3. The bacterium according to claim 2, wherein the expression of one or more of the genes is enhanced by increasing the copy number of the gene or genes, or modifying an expression control sequence of the gene or genes so that the expression of the gene or genes is enhanced.

4. The bacterium according to claim 3, wherein the copy number is increased by transformation of the bacterium with a low copy vector containing the gene or genes.

5. The bacterium according to claim 2 wherein the genes are originated from a bacterium belonging to the genus Escherichia.

6. The bacterium according to claim 1, wherein the bacterium has been further modified to enhance expression of one or more genes selected from the group consisting of the mutant thrA gene which codes for aspartokinase homoserine dehydrogenase I resistant to feed back inhibition by threonine, the thrB gene which codes for homoserine kinase, the thrC gene which codes for threonine synthase, and the rhtA gene, which codes for a putative transmembrane protein.

7. The bacterium according to claim 5, wherein the bacterium has been further modified to enhance expression of one or more genes selected from the group consisting of the mutant thrA gene which codes for aspartokinase homoserine dehydrogenase I resistant to feed back inhibition by threonine, the thrB gene which codes for homoserine kinase, the thrC gene which codes for threonine synthase, and the rhtA gene which codes for a putative transmembrane protein.

8. The bacterium according to claim 6, wherein the bacterium has been modified to increase the expression amounts of the mutant thrA gene, the thrB gene, the thrC gene and the rhtA gene.

9. A method for producing L-threonine comprising cultivating the bacterium of claim 1 in a culture medium to produce and cause accumulation of L-threonine in the culture medium, and collecting the L-threonine from the culture medium.

10. A method for producing L-threonine comprising cultivating the bacterium of claim 2 in a culture medium to produce and cause accumulation of L-threonine in the culture medium, and collecting the L-threonine from the culture medium.

11. The L-threonine-producing bacterium of claim 2, wherein the bacterium has been modified to enhance expression of the glk gene.

12. The L-threonine-producing bacterium of claim 2, wherein the bacterium has been modified to enhance expression of the pgi gene.

13. The L-threonine-producing bacterium of claim 2, wherein the bacterium has been modified to enhance expression of the pfkA gene.

14. The L-threonine-producing bacterium of claim 2, wherein the bacterium has been modified to enhance expression of the tpiA gene.

15. The L-threonine-producing bacterium of claim 2, wherein the bacterium has been modified to enhance expression of the gapA gene.

16. The L-threonine-producing bacterium of claim 2, wherein the bacterium has been modified to enhance expression of the pgk gene.

17. The L-threonine-producing bacterium of claim 2, wherein the bacterium has been modified to enhance expression of the eno gene.

18. The L-threonine-producing bacterium of claim 2, wherein the bacterium has been modified to enhance expression of the pykA gene.


SEQUENCE LISTING (34 sequences)

SEQ ID NO: 1 - DNA, 966 bp, Escherichia coli
  CDS (1)..(966)
atgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgatcccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggagcgcgtgctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaagcctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaa

SEQ ID NO: 2 - protein, 321 aa, Escherichia coli
MTKYALVGDVGGTNARLALCDIASGEISQAKTYSGLDYPSLEAVIRVYLEEHKVEVKDGCIAIACPITGDWVAMTNHTWAFSIAEMKKNLGFSHLEIINDFTAVSMAIPMLKKEHLIQFGGAEPVEGKPIAVYGAGTGLGVAHLVHVDKRWVSLPGEGGHVDFAPNSEEEAIILEILRAEIGHVSAERVLSGPGLVNLYRAIVKADNRLPENLKPKDITERALADSCTDCRRALSLFCVIMGRFGGNLALNLGTFGGVFIAGGIVPRFLEFFKASGFRAAFEDKGRFKEYVHDIPVYLIVHDNPGLLGSGAHLRQTLGHIL

SEQ ID NO: 3 - DNA, 1650 bp, Escherichia coli
  CDS (1)..(1650)
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 4 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 5 - DNA, 963 bp, Escherichia coli
  CDS (1)..(963)
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 6 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 7 - DNA, 768 bp, Escherichia coli
  CDS (1)..(768)
atgcgacatcctttagtgatgggtaactggaaactgaacggcagccgccacatggttcacgagctggtttctaacctgcgtaaagagctggcaggtgttgctggctgtgcggttgcaatcgcaccaccggaaatgtatatcgatatggcgaagcgcgaagctgaaggcagccacatcatgctgggtgcgcaaaacgtggacctgaacctgtccggcgcattcaccggtgaaacctctgctgctatgctgaaagacatcggcgcacagtacatcatcatcggtcactctgaacgtcgtacttaccacaaagaatctgacgaactgatcgcgaaaaaattcgcggtgctgaaagagcagggcctgactccggttctgtgcatcggtgaaaccgaagctgaaaatgaagcgggcaaaactgaagaagtttgcgcacgtcagatcgacgcggtactgaaaactcagggtgctgcggcattcgaaggtgcggttatcgcttacgaacctgtatgggcaatcggtactggcaaatctgcaactccggctcaggcacaggctgttcacaaattcatccgtgaccacatcgctaaagttgacgctaacatcgctgaacaagtgatcattcagtacggcggctctgtaaacgcgtctaacgctgcagaactgtttgctcagccggatatcgacggcgcgctggttggtggtgcttctctgaaagctgacgccttcgcagtaatcgttaaagctgcagaagcggctaaacaggcttaa

SEQ ID NO: 8 - protein, 255 aa, Escherichia coli
MRHPLVMGNWKLNGSRHMVHELVSNLRKELAGVAGCAVAIAPPEMYIDMAKREAEGSHIMLGAQNVDLNLSGAFTGETSAAMLKDIGAQYIIIGHSERRTYHKESDELIAKKFAVLKEQGLTPVLCIGETEAENEAGKTEEVCARQIDAVLKTQGAAAFEGAVIAYEPVWAIGTGKSATPAQAQAVHKFIRDHIAKVDANIAEQVIIQYGGSVNASNAAELFAQPDIDGALVGGASLKADAFAVIVKAAEAAKQA

SEQ ID NO: 9 - DNA, 996 bp, Escherichia coli
  CDS (1)..(996)
atgactatcaaagtaggtatcaacggttttggccgtatcggtcgcattgttttccgtgctgctcagaaacgttctgacatcgagatcgttgcaatcaacgacctgttagacgctgattacatggcatacatgctgaaatatgactccactcacggccgtttcgacggtaccgttgaagtgaaagacggtcatctgatcgttaacggtaaaaaaatccgtgttaccgctgaacgtgatccggctaacctgaaatgggacgaagttggtgttgacgttgtcgctgaagcaactggtctgttcctgactgacgaaactgctcgtaaacacatcaccgctggtgcgaagaaagtggttatgactggtccgtctaaagacaacactccgatgttcgttaaaggcgctaacttcgacaaatatgctggccaggacatcgtttccaacgcttcctgcaccaccaactgcctggctccgctggctaaagttatcaacgataacttcggcatcatcgaaggtctgatgaccaccgttcacgctactaccgctactcagaaaaccgttgatggcccgtctcacaaagactggcgcggcggccgcggcgcttcccagaacatcatcccgtcctctaccggtgctgctaaagctgtaggtaaagtactgccagaactgaatggcaaactgactggtatggcgttccgcgttccgaccccgaacgtatctgtagttgacctgaccgttcgtctggaaaaagctgcaacttacgagcagatcaaagctgccgttaaagctgctgctgaaggcgaaatgaaaggcgttctgggctacaccgaagatgacgtagtatctaccgatttcaacggcgaagtttgcacttccgtgttcgatgctaaagctggtatcgctctgaacgacaacttcgtgaaactggtatcctggtacgacaacgaaaccggttactccaacaaagttctggacctgatcgctcacatctccaaataa

SEQ ID NO: 10 - protein, 331 aa, Escherichia coli
MTIKVGINGFGRIGRIVFRAAQKRSDIEIVAINDLLDADYMAYMLKYDSTHGRFDGTVEVKDGHLIVNGKKIRVTAERDPANLKWDEVGVDVVAEATGLFLTDETARKHITAGAKKVVMTGPSKDNTPMFVKGANFDKYAGQDIVSNASCTTNCLAPLAKVINDNFGIIEGLMTTVHATTATQKTVDGPSHKDWRGGRGASQNIIPSSTGAAKAVGKVLPELNGKLTGMAFRVPTPNVSVVDLTVRLEKAATYEQIKAAVKAAAEGEMKGVLGYTEDDVVSTDFNGEVCTSVFDAKAGIALNDNFVKLVSWYDNETGYSNKVLDLIAHISK

SEQ ID NO: 11 - DNA, 1164 bp, Escherichia coli
  CDS (1)..(1164)
atgtctgtaattaagatgaccgatctggatcttgctgggaaacgtgtatttatccgtgcggatctgaacgtaccagtaaaagacgggaaagtaaccagcgacgcgcgtatccgtgcttctctgccgaccatcgaactggccctgaaacaaggcgcaaaagtgatggtaacttcccacctgggtcgtcctaccgaaggcgagtacaacgaagaattctctctgctgccggttgttaactacctgaaagacaaactgtctaacccggttcgtctggttaaagattacctcgacggcgttgacgttgctgaaggtgaactggttgttctggaaaacgttcgcttcaacaaaggcgagaagaaagacgacgaaaccctgtccaaaaaatacgctgcactgtgtgacgtgttcgtaatggacgcattcggtactgctcaccgcgcgcaggcttctactcacggtatcggtaaattcgctgacgttgcgtgcgcaggcccgctgctggcagctgaactggacgcgctgggtaaagcactgaaagaacctgctcgcccgatggtggctatcgttggtggttctaaagtatctaccaaactgaccgttctggactccctgtctaaaatcgctgaccagctgattgttggtggtggtatcgctaacacctttatcgcggcacaaggccacgatgtgggtaaatccctgtacgaagctgacctggttgacgaagctaaacgtctgctgaccacctgcaacatcccggttccgtctgatgttcgcgtagcaaccgagttctctgaaactgcaccggctaccctgaaatctgttaacgatgtgaaagctgacgagcagatcctggatatcggtgatgcttccgctcaggaactggctgaaatcctgaagaatgcgaaaaccattctgtggaacggtccggttggcgtgttcgaattcccgaacttccgcaaaggtactgaaatcgtggctaacgctatcgcagacagcgaagcgttctccatcgctggcggcggcgacactctggcagcaatcgacctgttcggcattgctgacaaaatctcctacatctccactggcggcggcgcattcctcgaattcgtggaaggtaaagtactgcctgcagtagcgatgctcgaagagcgcgctaagaagtaa

SEQ ID NO: 12 - protein, 387 aa, Escherichia coli
MSVIKMTDLDLAGKRVFIRADLNVPVKDGKVTSDARIRASLPTIELALKQGAKVMVTSHLGRPTEGEYNEEFSLLPVVNYLKDKLSNPVRLVKDYLDGVDVAEGELVVLENVRFNKGEKKDDETLSKKYAALCDVFVMDAFGTAHRAQASTHGIGKFADVACAGPLLAAELDALGKALKEPARPMVAIVGGSKVSTKLTVLDSLSKIADQLIVGGGIANTFIAAQGHDVGKSLYEADLVDEAKRLLTTCNIPVPSDVRVATEFSETAPATLKSVNDVKADEQILDIGDASAQELAEILKNAKTILWNGPVGVFEFPNFRKGTEIVANAIADSEAFSIAGGGDTLAAIDLFGIADKISYISTGGGAFLEFVEGKVLPAVAMLEERAKK

SEQ ID NO: 13 - DNA, 1299 bp, Escherichia coli
  CDS (1)..(1299)
atgtccaaaatcgtaaaaatcatcggtcgtgaaatcatcgactcccgtggtaacccgactgttgaagccgaagtacatctggagggtggtttcgtcggtatggcagctgctccgtcaggtgcttctactggttcccgtgaagctctggaactgcgcgatggcgacaaatcccgtttcctgggtaaaggcgtaaccaaagctgttgctgcggtaaacggcccgatcgctcaggcgctgattggcaaagatgctaaagatcaggctggcattgacaagatcatgatcgacctggacggcaccgaaaacaaatccaaattcggcgcgaacgcaatcctggctgtatctctggctaacgccaaagctgctgcagctgctaaaggtatgccgctgtacgagcacatcgctgaactgaacggtactccgggcaaatactctatgccggttccgatgatgaacatcatcaacggtggtgagcacgctgacaacaacgttgatatccaggaattcatgattcagccggttggcgcgaaaactgtgaaagaagccatccgcatgggttctgaagttttccatcacctggcaaaagttctgaaagcgaaaggcatgaacactgctgttggtgacgaaggtggctatgcgccgaacctgggttccaacgctgaagctctggctgttatcgctgaagctgttaaagctgctggttatgaactgggcaaagacatcactttggcgatggactgcgcagcttctgaattctacaaagatggtaaatacgttctggctggcgaaggcaacaaagcgttcacctctgaagaattcactcacttcctggaagaactgaccaaacagtacccgatcgtttctatcgaagacggtctggacgaatctgactgggacggtttcgcataccagaccaaagttctgggcgacaaaatccagctggttggtgacgacctgttcgtaaccaacaccaagatcctgaaagaaggtatcgaaaaaggtatcgctaactccatcctgatcaaattcaaccagatcggttctctgaccgaaactctggctgcaatcaagatggcgaaagatgctggctacactgcagttatctctcaccgttctggcgaaactgaagacgctaccatcgctgacctggctgttggtactgctgcaggccagatcaaaactggttctatgagccgttctgaccgtgttgctaaatacaaccagctgattcgtatcgaagaagctctgggcgaaaaagcaccgtacaacggtcgtaaagagatcaaaggccaggcataa

SEQ ID NO: 14 - protein, 432 aa, Escherichia coli
MSKIVKIIGREIIDSRGNPTVEAEVHLEGGFVGMAAAPSGASTGSREALELRDGDKSRFLGKGVTKAVAAVNGPIAQALIGKDAKDQAGIDKIMIDLDGTENKSKFGANAILAVSLANAKAAAAAKGMPLYEHIAELNGTPGKYSMPVPMMNIINGGEHADNNVDIQEFMIQPVGAKTVKEAIRMGSEVFHHLAKVLKAKGMNTAVGDEGGYAPNLGSNAEALAVIAEAVKAAGYELGKDITLAMDCAASEFYKDGKYVLAGEGNKAFTSEEFTHFLEELTKQYPIVSIEDGLDESDWDGFAYQTKVLGDKIQLVGDDLFVTNTKILKEGIEKGIANSILIKFNQIGSLTETLAAIKMAKDAGYTAVISHRSGETEDATIADLAVGTAAGQIKTGSMSRSDRVAKYNQLIRIEEALGEKAPYNGRKEIKGQA

SEQ ID NO: 15 - DNA, 1443 bp, Escherichia coli
  CDS (1)..(1443)
atgtccagaaggcttcgcagaacaaaaatcgttaccacgttaggcccagcaacagatcgcgataataatcttgaaaaagttatcgcggcgggtgccaacgttgtacgtatgaacttttctcacggctcgcctgaagatcacaaaatgcgcgcggataaagttcgtgagattgccgcaaaactggggcgtcatgtggctattctgggtgacctccaggggcccaaaatccgtgtatccacctttaaagaaggcaaagttttcctcaatattggggataaattcctgctcgacgccaacctgggtaaaggtgaaggcgacaaagaaaaagtcggtatcgactacaaaggcctgcctgctgacgtcgtgcctggtgacatcctgctgctggacgatggtcgcgtccagttaaaagtactggaagttcagggcatgaaagtgttcaccgaagtcaccgtcggtggtcccctctccaacaataaaggtatcaacaaacttggcggcggtttgtcggctgaagcgctgaccgaaaaagacaaagcagacattaagactgcggcgttgattggcgtagattacctggctgtctccttcccacgctgtggcgaagatctgaactatgcccgtcgcctggcacgcgatgcaggatgtgatgcgaaaattgttgccaaggttgaacgtgcggaagccgtttgcagccaggatgcaatggatgacatcatcctcgcctctgacgtggtaatggttgcacgtggcgacctcggtgtggaaattggcgacccggaactggtcggcattcagaaagcgttgatccgtcgtgcgcgtcagctaaaccgagcggtaatcacggcgacccagatgatggagtcaatgattactaacccgatgccgacgcgtgcagaagtcatggacgtagcaaacgccgttctggatggtactgacgctgtgatgctgtctgcagaaactgccgctgggcagtatccgtcagaaaccgttgcagccatggcgcgcgtttgcctgggtgcggaaaaaatcccgagcatcaacgtttctaaacaccgtctggacgttcagttcgacaatgtggaagaagctattgccatgtcagcaatgtacgcagctaaccacctgaaaggcgttacggcgatcatcaccatgaccgaatcgggtcgtaccgcgctgatgacctcccgtatcagctctggtctgccaattttcgccatgtcgcgccatgaacgtacgctgaacctgactgctctctatcgtggcgttacgccggtgcactttgatagcgctaatgacggcgtagcagctgccagcgaagcggttaatctgctgcgcgataaaggttacttgatgtctggtgacctggtgattgtcacccagggcgacgtgatgagtaccgtgggttctactaataccacgcgtattttaacggtagagtaa

SEQ ID NO: 16 - protein, 480 aa, Escherichia coli
MSRRLRRTKIVTTLGPATDRDNNLEKVIAAGANVVRMNFSHGSPEDHKMRADKVREIAAKLGRHVAILGDLQGPKIRVSTFKEGKVFLNIGDKFLLDANLGKGEGDKEKVGIDYKGLPADVVPGDILLLDDGRVQLKVLEVQGMKVFTEVTVGGPLSNNKGINKLGGGLSAEALTEKDKADIKTAALIGVDYLAVSFPRCGEDLNYARRLARDAGCDAKIVAKVERAEAVCSQDAMDDIILASDVVMVARGDLGVEIGDPELVGIQKALIRRARQLNRAVITATQMMESMITNPMPTRAEVMDVANAVLDGTDAVMLSAETAAGQYPSETVAAMARVCLGAEKIPSINVSKHRLDVQFDNVEEAIAMSAMYAANHLKGVTAIITMTESGRTALMTSRISSGLPIFAMSRHERTLNLTALYRGVTPVHFDSANDGVAAASEAVNLLRDKGYLMSGDLVIVTQGDVMSTVGSTNTTRILTVE

SEQ ID NO: 17 - DNA, 33 bp, Artificial Sequence
  Description of Artificial Sequence primer
ataggatccatgacaaagtatgcattagtcggt

SEQ ID NO: 18 - DNA, 33 bp, Artificial Sequence
  Description of Artificial Sequence primer
atagagctcgatttacagaatgtgacctaaggt

SEQ ID NO: 19 - DNA, 36 bp, Artificial Sequence
  Description of Artificial Sequence primer
tacattggatccatgattaagaaaatcggtgtgttg

SEQ ID NO: 20 - DNA, 34 bp, Artificial Sequence
  Description of Artificial Sequence primer
atcattgtcgacttaatacagttttttcgcgcag

SEQ ID NO: 21 - DNA, 30 bp, Artificial Sequence
  Description of Artificial Sequence primer
ataggatccatggtgagcgaacgcagacgc

SEQ ID NO: 22 - DNA, 30 bp, Artificial Sequence
  Description of Artificial Sequence primer
tttgagctcttacagaacgtcgatcgcgtt

SEQ ID NO: 23 - DNA, 45 bp, Artificial Sequence
  Description of Artificial Sequence primer
aaaggatccaagcttaaggagaaattaaaatgcgacatcctttag

SEQ ID NO: 24 - DNA, 51 bp, Artificial Sequence
  Description of Artificial Sequence primer
aaagagctcgaattcgtcgacagatctttattaagcctgtttagccgcttc

SEQ ID NO: 25 - DNA, 46 bp, Artificial Sequence
  Description of Artificial Sequence synthetic promoter Pa3m
aattcgtggtttaccacatgaagtaagacggtataatgtaccacag

SEQ ID NO: 26 - DNA, 46 bp, Artificial Sequence
  Description of Artificial Sequence synthetic promoter Pa3m
gcaccaaatggtgtacttcattctgccatattacatggtgtcctag

SEQ ID NO: 27 - DNA, 36 bp, Artificial Sequence
  Description of Artificial Sequence primer
ttaggatccttttattcactaacaaatagctggtgg

SEQ ID NO: 28 - DNA, 37 bp, Artificial Sequence
  Description of Artificial Sequence primer
ccgtctagaaatgaggtccagttcatccagtttacga

SEQ ID NO: 29 - DNA, 33 bp, Artificial Sequence
  Description of Artificial Sequence primer
actggatccatgtccaaaatcgtaaaaatcatc

SEQ ID NO: 30 - DNA, 28 bp, Artificial Sequence
  Description of Artificial Sequence primer
taagagctcttatgcctggcctttgatc

SEQ ID NO: 31 - DNA, 37 bp, Artificial Sequence
  Description of Artificial Sequence primer
aaaggatccggaggattgctaatgaaaaacatcaatc

SEQ ID NO: 32 - DNA, 29 bp, Artificial Sequence
  Description of Artificial Sequence primer
aaagagctcattaaccgcgccacgcttta

SEQ ID NO: 33 - DNA, 30 bp, Artificial Sequence
  Description of Artificial Sequence primer
ataggatccatgtctgtaattaagatgacc

SEQ ID NO: 34 - DNA, 30 bp, Artificial Sequence
  Description of Artificial Sequence primer
atagagctcttacttcttagcgcgctcttc